(19) United States
(12) Patent Application Publication
(10) Pub. No.: US 2004/0022665 A1
(43) Pub. Date: Feb. 5, 2004

(54) Mail box processor

(76) Inventor: Michael Yuan Lu, Lexington, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/306,774

(22) Filed: Nov. 26, 2002

Related U.S. Application Data
(60) Provisional application No. 60/333,443, filed on Nov. 26, 2001.

(51) Int. Cl. A01N 1/00

(52) U.S. Cl. 422/1; 422/292; 422/3

(57) ABSTRACT

A system and method of disinfecting and/or decontaminating articles such as pieces of mail that have been exposed to diverse biological and/or chemical contaminants. The system includes an enclosure having a door, an input port, and an output port, a mail tumbling drum, a radiation beam source and applicator, an electromagnetic field source and applicator, an ultraviolet radiation source and applicator, a chemical decontamination unit, and a status indicator. The enclosure door is opened, a quantity of mail is placed in the tumbling drum inside the enclosure, and the door is closed. At the end of a predetermined delay time, radiation beams, electromagnetic fields, ultraviolet radiation, and chemical decontaminates are applied to the mail for a predetermined time, and in predetermined combinations and sequences, thereby destroying essentially all biological viruses, bacteria, spores, pollutants, and bomb material that may be on or within the pieces of mail. The mail box processor is configured to minimize leakage so that contaminating substances harmful to humans and animals are contained and deactivated within the enclosure.

### CROSS REFERENCE TO RELATED APPLICATIONS

This application claims priority of U.S. Provisional Patent Application No. 60/333,443 filed Nov. 26, 2001 entitled MAIL BOX PROCESSOR.

### STATEMENT REGARDING FEDERALLY SPONSORED RESEARCH OR DEVELOPMENT

N/A

### BACKGROUND OF THE INVENTION

The present invention relates generally to systems and methods of disinfecting and/or decontaminating articles, and more specifically to a system and method of efficiently disinfecting and/or decontaminating articles such as pieces of mail that may have been exposed to diverse biological and/or chemical contaminants.

In recent years, there has been an increasing need for improved techniques of disinfecting and/or decontaminating articles that may have been intentionally or accidentally exposed to biological and/or chemical contaminants harmful to humans or animals. For example, such articles may have been inadvertently tainted with biological and/or chemical contaminants as a result of a laboratory or industrial accident. Alternatively, such articles may have been intentionally contaminated with harmful substances during the commission of a criminal or terrorist act.

Specifically, there is an increasing need for improved techniques of disinfecting and/or decontaminating articles that are shipped through the mail. This is because contaminated pieces of mail not only have the potential of harming the intended recipients of the mail and possibly those in the proximity of the intended recipients, but they can also harm significant numbers of other individuals such as postal employees who handle the contaminated mail as it passes through the postal system.

For example, the U.S. Postal Service has recently confronted the problem of handling letters that were contaminated with anthrax. Not only were recipients of the contaminated mail exposed to harmful anthrax spores, but numerous postal employees were also exposed to the anthrax spores leaking from the tainted letters, resulting in sickness, and in some cases, death. Further, significant numbers of people at the point of delivery of the contaminated letters were exposed to the anthrax. Because the anthrax spores released from the letters were transmitted through the air, entire buildings were contaminated by the spores via the buildings' heating and ventilation systems, resulting in the buildings' occupants being treated with powerful antibiotics to ward off anthrax-related illnesses. Moreover, because the anthrax-tainted letters contaminated some mail handling equipment at U.S. Post Offices, other mail passing through the postal system was tainted with the anthrax by cross-contamination, resulting in additional illness and deaths. Beyond the human toll, buildings and mail handling equipment were subjected to very costly decontamination procedures to remove the potentially harmful anthrax spores.

One way of guarding against contaminated articles from being shipped through the mail is to inspect each and every piece of mail at the point of entry into the postal system. However, this approach is generally regarded as unworkable because the U.S. Postal Service is estimated to handle hundreds of millions of pieces of mail each day. Further, the U.S. Postal Service currently has fewer than 2,000 postal inspectors charged with the task of investigating the misuse of the mail. Clearly, inspecting each piece of mail that passes through the postal system with such limited resources is virtually an insurmountable task.

Another approach to disinfecting and/or decontaminating pieces of mail is to irradiate the mail using electron beam technology. For example, bulk quantities of the mail may be irradiated by beams of high-energy electrons generated by an electron gun. Such technology has been employed to kill bacteria in food, and similar technology has also been employed to kill bacteria such as anthrax on or within pieces of mail.

However, this approach also has drawbacks in that such irradiation equipment has traditionally been costly. Moreover, the effectiveness of such irradiation equipment has been limited because articles such as pieces of mail may become contaminated with one or more of a variety of biological and/or chemical agents. For example, although irradiation equipment employing electron beam technology may be effective in killing anthrax spores, it may be incapable of destroying other biological contaminants such as HIV and E-Coli, and agents that cause, e.g., smallpox, influenza, plague, and botulism.

It would therefore be desirable to have a system and method of disinfecting and/or decontaminating articles such as pieces of mail. Such a system would be effective for disinfecting and/or decontaminating articles that have been exposed to diverse biological and/or chemical contaminants. It would also be desirable to have a disinfecting and/or decontaminating system that is compact, easy to use, and relatively low cost.

### BRIEF SUMMARY OF THE INVENTION

In accordance with the present invention, a system and method is disclosed that is capable of disinfecting and/or decontaminating articles such as pieces of mail that have been exposed to diverse biological and/or chemical contaminants. The presently disclosed system employs various technologies such as radiation beam technology, electromagnetic field technology, ultraviolet radiation technology, chemical decontamination technology, and suitable combinations of these technologies to provide effective disinfection and/or decontamination of mail at the point of entry into the postal system and/or at the point of mail delivery.

In one embodiment, the system for disinfecting and/or decontaminating articles such as pieces of mail comprises a mail box processor including an enclosure having a door, at least one input port, and at least one output port, a mail tumbling drum, at least one radiation beam source and applicator, at least one electromagnetic field source and applicator, at least one ultraviolet radiation source and applicator, at least one chemical decontamination unit, and a status indicator.

In the presently disclosed embodiment, the enclosure door is opened, a quantity of mail including suitably sized letters and packages is placed in the mail tumbling drum inside the enclosure, and the door is closed. The status indicator then flashes a warning light indicating that the disinfection/decontamination process is to begin within a predetermined delay time. At the end of the predetermined delay time, radiation beams, electromagnetic fields, ultraviolet radiation, and chemical decontaminates are applied to the quantity of mail in the tumbling drum for a predetermined time, and in predetermined combinations and sequences. Further, the mail tumbling drum rotates at predetermined speeds and directions to assure that each piece of mail is fully exposed to the beams, fields, radiation, and chemical decontaminates, thereby destroying essentially all biological viruses, bacteria, spores, pollutants, and bomb material that may be on or within the pieces of mail. The input and output ports of the mail box processor enclosure are configured to minimize leakage so that contaminating substances harmful to humans and animals are contained and deactivated within the enclosure.

By providing a mail box processor that employs technologies such as radiation beam, electromagnetic field, ultraviolet radiation, and chemical decontamination technologies for disinfecting and/or decontaminating pieces of mail within a secure enclosure, harmful substances including diverse biological and/or chemical contaminants on or within the mail can be deactivated while minimizing health risks to individuals in the proximity of the device.

Other features, functions, and aspects of the invention will be evident from the Detailed Description of the Invention that follows.

### BRIEF DESCRIPTION OF THE SEVERAL VIEWS OF THE DRAWING

The invention will be more fully understood with reference to the following Detailed Description of the Invention in conjunction with the drawings of which:

FIG. 1 is a perspective view of a mail box processor in accordance with the present invention;

FIG. 2 is a block diagram of the mail box processor of FIG. 1;

FIG. 3 is a schematic diagram of a power unit included in the mail box processor of FIG. 1; and

FIG. 4 is a flow diagram of a method of operation of the mail box processor of FIG. 1.

### DETAILED DESCRIPTION OF THE INVENTION

U.S. Provisional Patent Application No. 60/333,443 filed Nov. 26, 2001 entitled MAIL BOX PROCESSOR is incorporated herein by reference.

A system and method of disinfecting and/or decontaminating articles such as pieces of mail is provided that can be deployed at the point of entry into the postal system, at the point of mail delivery, and/or at any other suitable location. The system for disinfecting and/or decontaminating articles comprises a mail box processor that employs various technologies such as radiation beam technology, electromagnetic field technology, ultraviolet radiation technology, chemical decontamination technology, and suitable combinations thereof to disinfect/decontaminate the mail, while minimizing health risks to the intended mail recipients and individuals in the proximity of the device.

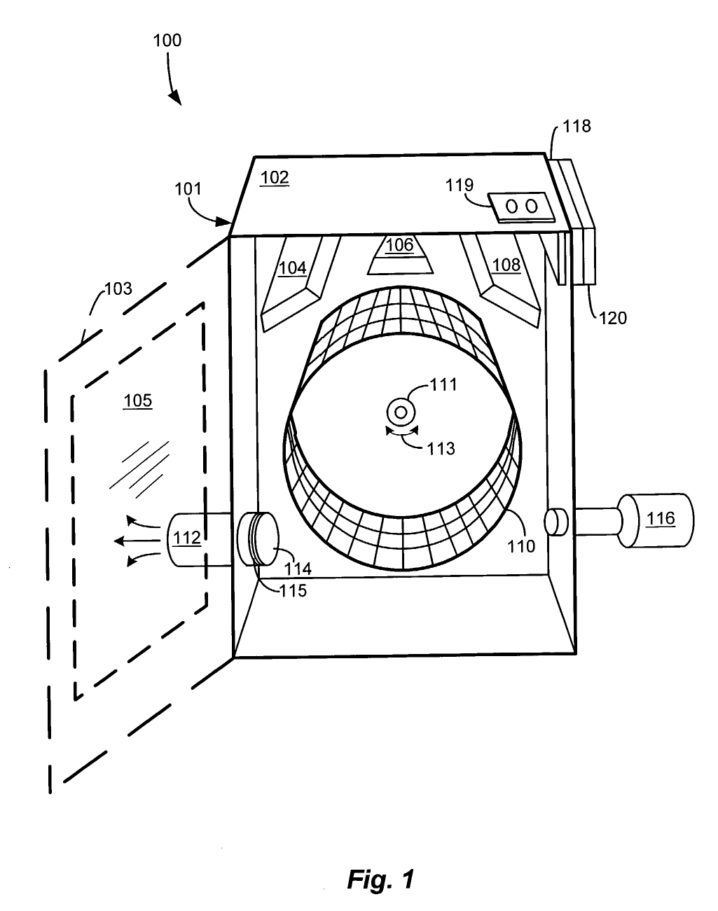

FIG. 1 depicts an illustrative embodiment of a system for disinfecting and/or decontaminating mail, in accordance with the present invention. In the illustrated embodiment, the system 100 comprises a mail box processor 101 including an enclosure 102 with a door 103 (shown in phantom for clarity of illustration), a mail tumbling drum 110, a decontamination process in-7 progress/completed status indicator 119, an input port 120, and an output port 112. The mail box processor 101 further includes a radiation beam source and applicator 104, an electromagnetic field source and applicator 106, an ultraviolet (“UV”) radiation source and applicator 108, and a chemical decontamination unit 116. It is understood, however, that in alternative embodiments, the mail box processor 101 may employ any other suitable disinfection/decontamination technology such as x-ray, gamma ray, broadband light beam, and oxidation technologies.

In the presently disclosed embodiment, the mail box processor 101 employs radiation beam technology, electromagnetic field technology, UV radiation technology, chemical decontamination technology, and/or suitable combinations of these technologies, for effectively disinfecting and/or decontaminating pieces of mail. To that end, a quantity of potentially contaminated mail is placed and confined in the mail tumbling drum 110 inside the enclosure 102, the enclosure door 103 is closed, and the mail in the tumbling drum 110 undergoes at least one disinfection/decontamination cycle using one or more of the above-mentioned technologies.

It is noted that the enclosure 102 including the door 103 is suitably shielded and gasketed to prevent leakage of electromagnetic and/or UV radiation during the disinfection/decontamination cycle. The enclosure 102 is further configured to prevent potentially harmful biological and/or chemical substances from escaping until the substances are either destroyed or otherwise rendered inactive by the decontamination process. As shown in FIG. 1, the enclosure door 103 includes a transparent section 105 to allow a human operator to observe the mail articles in the tumbling drum 110.

In the illustrated embodiment, the mail tumbling drum 110 is configured to allow radiation beams applied by the radiation beam applicator 104, electromagnetic fields applied by the electromagnetic field applicator 106, UV radiation applied by the UV radiation applicator 108, and chemical decontaminates applied by the chemical decontamination unit 116 to impinge upon the mail in the tumbling drum 110. For example, the mail tumbling drum 110 may have a mesh construction with suitably sized holes (not numbered). It is understood that the pieces of mail placed in the tumbling drum 110 include letters, packages, etc., suitably sized for placement and retention in the drum.

In the preferred embodiment, the mail tumbling drum 110 can handle at least 30 lbs. of mail during each disinfection/decontamination cycle. Further, the tumbling drum 110 is configured for rotationally oscillating about a hub 111, as depicted by directional arrows 113. The speed and direction of rotation of the mail tumbling drum 110 can be pre-set, e.g., pre-programmed, to assure that all portions of the mail are exposed to the applied radiation, electromagnetic fields, and/or chemical decontaminates. For example, the speed may be pre-set to a single speed, or pre-programmed to a number of varying speeds. Similarly, the direction of rotation may be pre-set to a single rotation direction, or pre-programmed to change direction a predetermined number of times. Moreover, all surfaces of the mail tumbling drum 110, and all internal surfaces of the enclosure 102 including the door 103, are preferably highly reflective to amplify the light ray disinfection energy applied to the mail during the decontamination process.

As described above, the enclosure 102 is configured to prevent potentially harmful biological and/or chemical substances (e.g., bacteria, bacteria spores, viral particles, and agents carrying viruses) inside the enclosure from escaping. To that end, the air pressure inside the enclosure 102 is made to be below atmospheric pressure. Specifically, the input port 120 is configured to allow ambient air to pass therethrough, and to enter the enclosure 102 via one or more orifices (not numbered). It is noted that a filter 118 may be employed to filter the ambient air before it enters the enclosure 102. The output port 112 is configured to draw the ambient air from the input port 120, through the inside of the enclosure 102, and back outside the enclosure 102, using, e.g., an air blower (not shown). As a result, even if there were any unwanted air leaks in the system 100, the air would simply be drawn into the enclosure 102 to be subsequently expelled through the output port 112.

As shown in FIG. 1, before the air inside the enclosure 102 re-enters the ambient environment via the output port 112, the air first passes through a filter 114, which in the presently disclosed embodiment is configured for capturing particulate matter. In the preferred embodiment, the first filter is a High Efficiency Particle Air (HEPA) filter capable of removing particles as small as approximately 1 pm from the air. A paper dust guard (not shown) may be disposed in front of the HEPA filter 114 to block any paper dust particles that may have released from the mail in the tumbling drum 110, thereby preventing the paper dust from filling the HEPA filter 114. Next, the air passes through a second filter 115, which is preferably a chemical filter capable of extracting desorbed chemicals from the air before it is expelled through the output port 112.

In the presently disclosed embodiment, the HEPA filter 114 and the chemical filter 115 are disposed within the enclosure 102 so that both of the filters 114-115 are exposed to the radiation beams, electromagnetic fields, UV radiation, and chemical decontaminates applied by the radiation beam applicator 104, the electromagnetic field applicator 106, the UV radiation applicator 108, and the chemical decontamination unit 116, respectively. In this way, the HEPA filter 114 and the chemical filter 115 are disinfected/decontaminated along with the mail during the decontamination process.

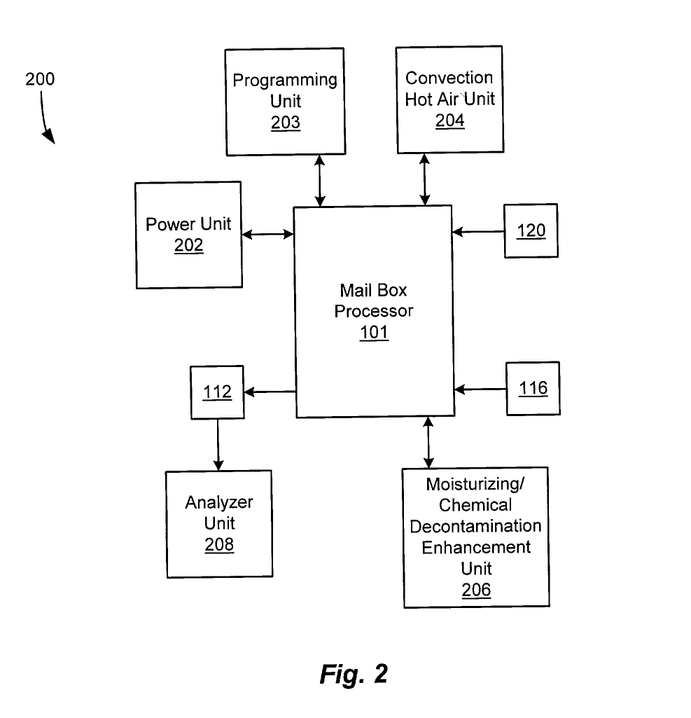

FIG. 2 depicts a block diagram 200 of the system 100 for disinfecting and/or decontaminating mail (see FIG. 1). As shown in FIG. 2, the system 200 includes the mail box processor 101, the input and output ports 120 and 112, and the chemical decontamination unit 116. The system 200 further includes a power unit 202, a programming unit 203, a convection hot air unit 204, a moisturizing/chemical decontamination enhancement unit 206, and an analyzer unit 208.

In the preferred embodiment, the output port 112 includes a quartz tube (not numbered) through which the air inside the enclosure 102 (see FIG. 1) is expelled to the ambient environment. The analyzer unit 208 (see FIG. 2) is preferably operatively connected to the quartz tube for analyzing the expelled air to detect any harmful biological and/or chemical substances that might inadvertently escape from the mail box processor 101 during the decontamination process. For example, the quartz tube may be surrounded by an electromagnetic field to keep molecules within the tube suspended, thereby aiding in the subsequent analysis of the expelled air by the analyzer unit 208. Further, the analyzer unit 208 may employ one or more algorithms for removing background noise from selected DNA/RNA signals of specific molecular weights to aid in determining the species and origin of detected biological substances.

The convection hot air unit 204 is employed in conjunction with the input port 120 for optionally pre-heating the ambient air being drawn into the mail box processor 101. In alternative embodiments, the convection hot air unit 204 is also configured to provide infrared radiation disinfection capabilities that may be employed in conjunction with the radiation beam, electromagnetic field, and/or UV radiation applicators 104, 106, and 108 (see FIG. 1). For example, the electromagnetic field source and applicator 106 may be configured to apply microwave energy to the potentially contaminated mail in the tumbling drum 110. Because the mail may include metal objects such as staples or paper clips, the microwave energy and the infrared energy may be alternately applied to the mail by the electromagnetic field applicator 106 and the convection hot air unit 204, respectively, to reduce the chance of fire, which might occur if the microwave energy were continuously applied to the stapled pieces of mail during a typical decontamination process lasting 1-30 minutes. Further, by periodically pausing the application of the microwave energy, the power requirements of the mail box processor 101 can be reduced.

The moisturizing/chemical decontamination enhancement unit 206 is employed in conjunction with the chemical decontamination unit 116 (see FIG. 1) to produce an optimal disinfection chemical/moisture-based environment inside the mail box processor 101, thereby improving the effectiveness of the chemical decontamination portion of the disinfection/decontamination process. It is noted that the moisturizing/chemical decontamination enhancement unit 206 may also be employed to inject suitable chemicals, gas, and/or moisture inside the enclosure 102 (see FIG. 1) to prevent overheating of the enclosure contents, and further reduce the chance of fire within the mail box processor 101. For example, the moisturizing/chemical decontamination enhancement unit 206 may inject a chemical operative to eliminate oxygen from the enclosure 102.

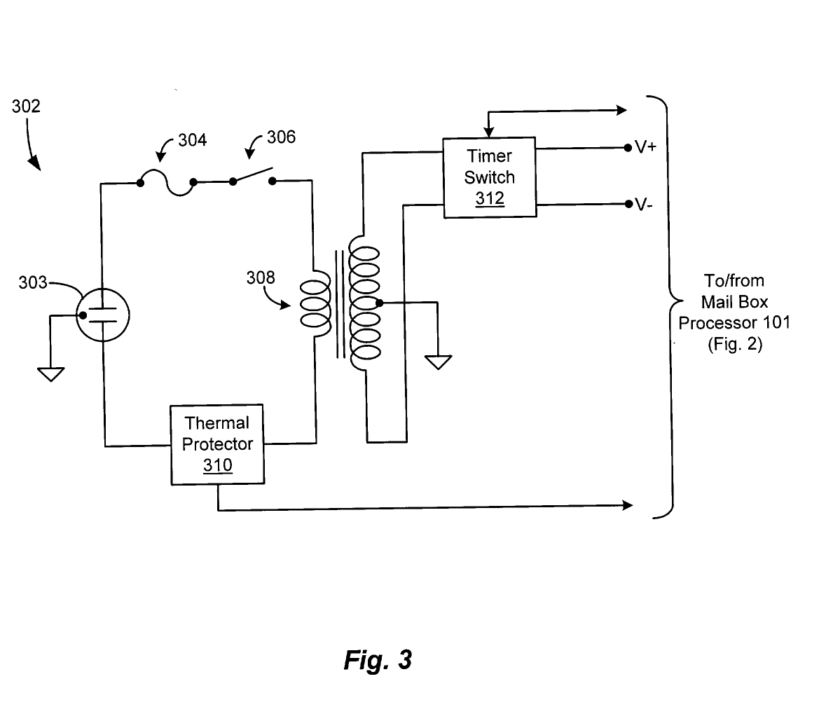

FIG. 3 depicts an illustrative embodiment 302 of the power unit 202 (see FIG. 2). In the illustrated embodiment, the power unit 302 includes a connection 303 to line power, a fuse 304, a power switch 306, a transformer 308, a thermal protector 310, and a timer switch 312. For example, the specifications for the line power may be approximately 25 A, 120 V. As described above, the moisturizing/chemical decontamination enhancement unit 206 may be employed to inject suitable chemicals, gas, and/or moisture inside the enclosure 102 (see FIG. 1) to prevent overheating of the enclosure contents. The thermal protector 310 is configured to disconnect the power from the mail box processor 101 in the event the temperature inside the enclosure 102 exceeds a predetermined level. For example, the thermal protector 310 may comprise one or more Resistance Thermal Detectors (RTDs). In the preferred embodiment, the thermal protector 310 is further configured to convey status information to the mail box processor 101. Moreover, the timer switch 312 is configurable to provide power to the mail box processor 101 via power connections V+ and V− after a predetermined delay time. For example, the predetermined delay time may be pre-programmed in the timer switch 312 via the programming unit 203. The timer switch 312 is further configured to convey status information to the mail box processor 101.

As further described above, the speed and direction of rotation of the mail tumbling drum 110 may be pre-programmed, and the delay time provided by the timer switch 312 (see FIG. 3) may also be pre-programmed. To that end, the programming unit 203 comprises a suitable user interface, processor, and memory to enable the human operator to program these desired settings. Further, the programming unit 203 may be employed to execute appropriate disinfection/decontamination applications to assure that the radiation beams, electromagnetic fields, UV radiation, and chemical decontaminates are applied to the mail in the most effective intensities, combinations, and/or sequences for killing/destroying biological and/or chemical substances on or within the mail. For example, an appropriate decontamination process may include selectively activating/deactivating the chemical decontamination unit 116 to inject ozone into the enclosure 102, and then activating/deactivating the UV radiation applicator 108 to apply UV radiation to kill harmful bacteria on the mail. It is understood that the radiation beam applicator 104, and the electromagnetic field applicator 106, may also be activated and controlled via the programming unit 203.

It should be appreciated that the radiation beam source and applicator 104 of the mail box processor 101 (see FIG. 1) may be configured to provide an electron beam, or any other suitable radiation beam, having an intensity sufficient to kill harmful biological contaminants in mail disposed in the mail box processor 101. Further, the electromagnetic field source and applicator 106 may be configured to provide microwave, Radio Frequency (RF) wave, or any other suitable electromagnetic energy, and the UV radiation source and applicator 108 may be configured to provide UV radiation in the UV-C band, or any other suitable type of UV radiation, to kill the biological contaminants. Moreover, the chemical decontamination unit 116 may be configured to apply any suitable chemical decontaminates to rid the mail of chemical contamination. For example, the chemical decontamination unit 116 may employ one or more chemical bags to facilitate the application of the chemical decontaminates. It is further appreciated that the mail box processor 101 may be employed for disinfecting/decontaminating pieces of mail or any other suitable article.

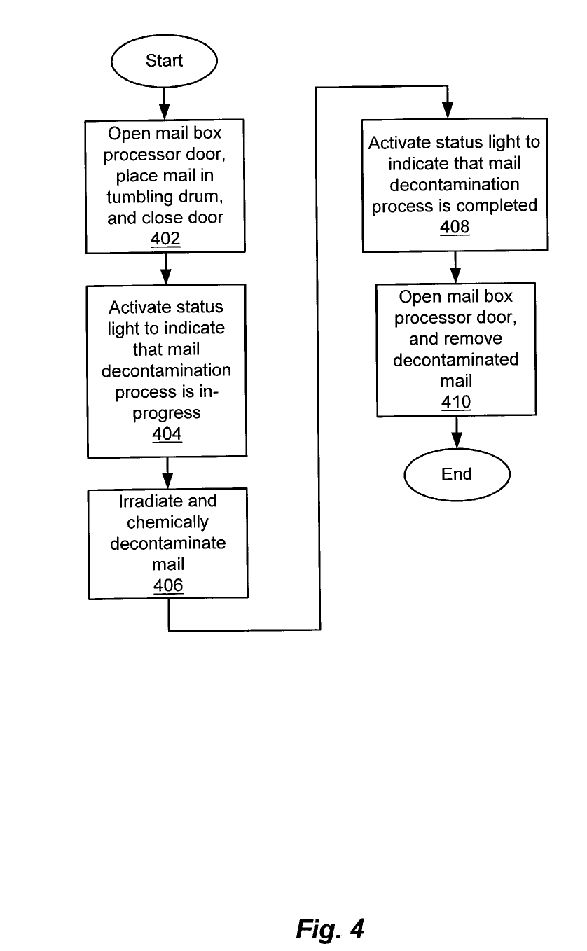

A method of operating the presently disclosed mail box processor 101 (see FIG. 1) is illustrated by reference to FIG. 4. As depicted in step 402, the door is opened, a quantity of mail is placed in the tumbling drum, and the door is closed. Next, the status indicator “in-progress” light is activated, as depicted in step 404, to alert individuals in the proximity of the mail box processor that the mail decontamination process will be in-progress after the pre-programmed delay time, if any. The mail is then irradiated and chemically decontaminated, as depicted in step 406, via the radiation beam applicator, the electromagnetic field applicator, the UV radiation applicator, and the chemical decontamination unit. It is appreciated that the mail box processor is pre-programmed to apply the radiation and chemical decontaminates in the most effective intensities, combinations, and/or sequences for eliminating biological and chemical contaminates from the mail. At the end of the decontamination cycle, the status indicator “completed” light is activated, as depicted in step 408, to provide notification that the mail decontamination process is completed. The door of the mail box processor is then opened, as depicted in step 410, and the decontaminated mail is removed.

It will further be appreciated by those of ordinary skill in the art that modifications to and variations of the above-described mail box processor may be made without departing from the inventive concepts disclosed herein. Accordingly, the invention should not be viewed as limited except as by the scope and spirit of the appended claims.

What is claimed is:

1. A system for decontaminating at least one article from at least one biological or chemical substance, comprising:
an enclosure having a door, the enclosure defining a decontamination region, the door being configured to close off the decontamination region, and to allow the at least one article to be disposed in and to be removed from the decontamination region;
a plurality of decontamination units operatively disposed in the decontamination region, each decontamination unit being operative to decontaminate the at least one article from at least one predetermined biological or chemical substance; and
a programming unit including at least one processor and at least one memory, the programming unit being communicably coupled to the plurality of decontamination units, the programming unit being operative to execute at least one application for selectively activating and deactivating the plurality of decontamination units.

2. The system of claim 1 wherein the programming unit is further operative to execute at least one application for activating selected decontamination units in at least one predetermined combination.

3. The system of claim 1 wherein the programming unit is further operative to execute at least one application for activating selected decontamination units in at least one predetermined sequence.

4. The system of claim 1 wherein the plurality of decontamination units include at least one of a radiation beam source and applicator, an electromagnetic field source and applicator, an ultraviolet radiation source and applicator, a chemical decontamination unit, and an infrared radiation source and applicator.

5. The system of claim 1 further including an article holding device operatively disposed in the decontamination region, and configured to cause the at least one article to tumble while being decontaminated by the plurality of decontamination units.

6. The system of claim 5 wherein the article holding device includes at least one wall having a mesh construction.

7. The system of claim 5 wherein the article holding device comprises a rotatable drum, and the programming unit is further operative to execute at least one application for rotating the drum at at least one predetermined speed.

8. The system of claim 5 wherein the article holding device comprises a rotatable drum, and the programming unit is further operative to execute at least one application for selectively changing the direction of rotation of the drum.

9. The system of claim 1 wherein the air pressure in the decontamination region is maintained at below atmospheric pressure.

10. The system of claim 1 wherein the enclosure has an input port configured to allow gas to enter the decontamination region, and an output port configured to allow gas to be expelled from the decontamination region.

11. The system of claim 10 wherein the input port comprises at least one filter.

12. The system of claim 10 wherein the output port comprises at least one filter.

13. The system of claim 12 wherein the at least one filter comprises a HEPA filter.

14. The system of claim 12 wherein the at least one filter comprises a chemical filter.

15. The system of claim 10 further including at least one analyzer unit operatively coupled to the output port, and configured to analyze the gas expelled via the output port.

16. The system of claim 10 further including a convection hot air unit configured to pre-heat the gas entering via the input port.

17. The system of claim 1 further including at least one moisturizing unit configured to inject moisturized gas into the decontamination region.

18. The system of claim 1 further including a power unit operatively coupled to the plurality of decontamination units and the programming unit, the power unit being configured to remove power from at least the plurality of decontamination units in the event the temperature in the decontamination region exceeds a predetermined level.

19. The system of claim 1 further including a power unit operatively coupled to the plurality of decontamination units and the programming unit, the power unit being configured to apply power to the plurality of decontamination units after the expiration of a programmable time.

20. A method of decontaminating at least one article from at least one biological or chemical substance, comprising the steps of:
disposing the at least one article in an enclosure, the enclosure having a door and defining a decontamination region; and
decontaminating the at least one article from the at least one biological or chemical substance by a plurality of decontamination units, the decontamination units being operatively disposed in the decontamination region,
wherein the decontaminating step includes selectively activating and deactivating the plurality of decontamination units by a programming unit executing at least one decontamination application.

21. The method of claim 20 wherein the decontaminating step includes activating selected decontamination units in at least one predetermined combination.

22. The method of claim 20 wherein the decontaminating step includes activating selected decontamination units in at least one predetermined sequence.

23. The method of claim 20 wherein the disposing step includes disposing the at least one article in a tumbling drum operatively disposed in the decontamination region, and the decontaminating step includes tumbling the at least one article by the tumbling drum.

24. The method of claim 20 further including the step of maintaining the air pressure in the decontamination region at below atmospheric pressure.

25. The method of claim 20 wherein the decontaminating step includes drawing gas into the decontamination region via an input port, and expelling gas from the decontamination region by via an output port.

26. The method of claim 25 further including the step of analyzing the expelled gas.

27. The method of claim 25 further including the step of filtering the expelled gas by at least one of a HEPA filter and a chemical filter.

28. The method of claim 25 further including the step of pre-heating the gas entering via the input port by a convection hot air unit.

29. The method of claim 20 further including the step of injecting moisturized gas into the decontamination region by a moisturizing unit.

30. The method of claim 20 further including the step of removing power from the plurality of decontamination units in the event the temperature in the decontamination region exceeds a predetermined level.

31. The method of claim 20 further including the step of applying power to the plurality of decontamination units after the expiration of a programmable time.
